(19) United States
(12) Patent Application Publication
(10) Pub. No.: US 2022/0175926 A1
(43) Pub. Date: Jun. 9, 2022

(54) TARGETED SYNERGISTIC CANCER IMMUNOTHERAPY

(71) Applicants: The Brigham and Women's Hospital, Inc., Boston, MA (US); Children's Medical Center Corporation, Boston, MA (US)

(72) Inventors: Jeffrey M. KARP, Brookline, MA (US); Rui KUAI, Brookline, MA (US); Jun XU, Roxbury, MA (US); Wenmin YUAN, Brookline, MA (US); Dania ZHIVAKI, Boston, MA (US); Jonathan C. KAGAN, Brookline, MA (US); Andrew BELLINGER, Boston, MA (US)

(21) Appl. No.: 17/598,620

(22) PCT Filed: Mar. 30, 2020
(86) PCT No.: PCT/US20/25704

§ 371 Date: Sep. 27, 2021

Related U.S. Application Data
(60) Provisional application No. 62/826,061, filed on Mar. 29, 2019.

(51) Int. Cl. A61K 41/13; A61K 45/06; A61P 35/00

(52) U.S. Cl. CPC A61K 41/13; A61P 35/00; A61K 45/06

(57) ABSTRACT

Reactive oxygen species (ROS) generated with noninvasive ultrasound and sonosensitizers, potently synergize with selected immunomodulators to hyperactivate dendritic cells and macrophages at desired locations and times within the body. Together with the tumor antigens provided by dying/dead tumor cells, these signals can result in activation of adaptive immune responses. This approach is useful for eliciting T cell responses within tumors present in any tissue of the body.

### CROSS-REFERENCE TO RELATED APPLICATIONS

This application claims the benefit of U.S. Provisional Application No. 62/826,061, filed Mar. 29, 2019, the disclosure of which is incorporated herein by reference in its entirety.

### BACKGROUND

One of the cancer immunotherapy goals is to elicit potent antitumor immune responses, especially T cell responses, which are the major driving forces to fight cancer. While multiple options are available to provide antigens for T cell activation, to improve therapeutic efficacy, approaches are needed to induce secretion of cytokines such as IL-1 that can promote T cell activation. Previously, proinflammatory adjuvants such as lipopolysaccharide (LPS) in combination with oxidized phospholipids (oxPAPC) have been used to induce IL-1 from macrophages or dendritic cells. The addition of IL-1β to the repertoire of immunomodulators secreted by these cells endows them with the ability to induce potent antigen specific T cells responses. Consequently, cells stimulated in this manner have been dubbed “hyperactive”, the activities of which may be critical to improve therapies designed to stimulate adaptive immunity. To date, technologies that hyperactive cells are restricted to those that are accessible by needle injections. A general approach to hyperactivate dendritic cells (or macrophages) in other tissues of the body remains to be developed.

Immunotherapy with immune checkpoint blockade (ICB) has achieved great initial success, as shown by the remarkable improvement on overall survival and durable responses for some patients treated with ICB (Ribas et al., Science. 359(6382):1350-1355 (2018)). However, the response rate is only ˜25% and can be even lower for certain cancers with low immunogenicity, thus making it urgent to improve the response rate of ICB (Sharma et al., Science. 348(6230):56-61 (2015)). Increasing evidence has indicated the response to ICB is positively correlated with infiltration of antitumor immune cells, especially T cells in the tumor microenvironment (TME) (Chen et al., Nature. 541(7637):321-330 (2017); Binnewies et al., Nat Med. 24(5):541-550 (2018); Fridman et al., Nat Rev Clin Oncol. 14(12):717-734 (2017).) Therefore, elicitation of potent T cell responses is critical to improve the response rate of ICB.

Vaccines such as peptide vaccines or mRNA vaccines have been used to induce potent T cell responses that can inhibit tumor growth and synergize with ICB (Kuai et al., Nat Mater. 16(4):489-496 (2017); Kranz et al., Nature. 534(7607):396-401 (2016)), but these approaches require the identification and use of tumor antigens. While analysis of tumor biopsy samples can facilitate identification of tumor neoantigens in some cases, it is invasive, low yield and technically challenging. Local injections of therapies into tumors can assist in inducing anti-tumor immune responses while preventing systemic immune response (Sagiv-Barfi et al., Sci Transl Med. 10(426), 2018), but non-invasive treatment approaches would be preferable.

Recently, tumor cells killed in situ with chemotherapy (Pfirschke et al., Immunity. 44(2):343-354 (2016)), irradiation therapy (Twyman-Saint et al., Nature. 520(7547):373-377 (2015)), photothermal therapy (Chen et al., Nat Commun. 7:13193 (2016)), photodynamic therapy (Castano et al., Nat Rev Cancer. 6(7):535-545 (2006)), or sonodynamic therapy (Nomikou et al., ChemMedChem. 7(8):1465-1471 (2012)) have been used to generate tumor antigens for dendritic cells (DCs) that present the antigen epitopes for T cell activation, but these approaches cannot control the generation of cytokines that have profound impact on the activation of T cells. For example, recent studies have shown higher levels of IL-1β secreted from macrophages or dendritic cells are correlated with stronger T cell responses. To induce IL-1β secretion, immuno-modulators such as oxidized 1-palmitoyl-2-arachidonoyl-sn-glycero-3-phosphocholine (oxPAPC) are combined with proinflammatory adjuvants such as lipopolysaccharide (LPS) (Zanoni et al., Science. 352(6290):1232-1236 (2016)), without which minimal IL-1β can be generated. These stimulations result in the “hyperactivation” of DCs and macrophages, resulting in stronger and more effective T cell responses than those elicited by standard LPS-based immunizations. However, the ability to hyperactivate DCs and macrophages is restricted to dermal and muscular cells (accessed by a needle injection). There is no current method to induce phagocyte hyperactivation in deeper tissues of the body. A general method to simultaneously achieve the in situ killing of tumor cells (to generate tumor antigens) and controlled generation of hyperactive cells is needed to overcome the limitations of currently available approaches.

Inspired by the fact that elevated reactive oxygen species (ROS) are correlated with increased activity of immune cells (Habtetsion et al., Cell Metab. 28(2):228-242 e226 (2018)), we set out to control the generation of ROS in order to induce the hyperactivation of immune cells and secretion of critical cytokines for T cell activation. In particular, we choose to generate ROS using ultrasound and sonosensitizers due to their good safety profiles (Rwei et al., Nat Biomed Eng. 1:644-653 (2017)), and applicability to a broad range of tissues, including those relatively deep tissues that are hard to reach with biopsy or lasers. When sonosensitizers are exposed to ultrasound with a certain frequency and intensity, the energy delivered by the sound wave can excite the sonosensitizers, which can generate ROS when the excited electron returns to the ground state. While this approach (also known as sonodynamic therapy) has been used to inhibit tumor growth in vitro and in vivo, how to use it to control the activation of immune cells, especially to control the secretion of critical cytokines from immune cells to promote T cell activation has not been thoroughly explored.

### SUMMARY

In one aspect, the invention provides a method of inducing cytokine secretion, the method including: (a) contacting mammalian antigen presenting cells (APCs) with a sonosensitizer and an immunomodulator; and (b) exposing the APCs of (a) to ultrasound radiation for a period of time sufficient to induce cytokine secretion by the APCs.

In some embodiments, the cytokine comprises one or both of IL-1β and TNF-α.

In some embodiments, the APCs comprise macrophages. In some embodiments, the APCs are present in a mammalian subject. In some embodiments, the mammalian subject has a tumor and contacting and exposing the APCs results in killing cells of the tumor.

In another aspect, the invention provides a method of inducing secretion of IL-1β in a mammalian subject comprising

(a) administering a sonosensitizer to the subject,

(b) administering an immunomodulator to the subject, and

(c) thereafter, exposing the subject to ultrasound radiation.

In some embodiments, the sonosensitizer comprises a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis (dithiolene) complex, bis (benzene-dithiolate) complex, iodoaniline dye, bis (S,O-dithiolene) complex, or a derivative or combination thereof.

In some embodiments, the sonosensitizer is encapsulated in a liposome. In some embodiments, the sonosensitizer isconjugated with a lipophilic moiety. In some embodiments, the lipophilic moiety is dioleoylphosphatidylethanolamine (DOPE) or cholesterol.

In some embodiments, the immunomodulator comprises 1-palmitoyl-2-arachidonoyl-sn-glycero-3-phosphocholine (PAPC), LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21, stimulon, vadimezan, AsA404 (DMXAA), a STING agonist (e.g., a cyclic dinucleotide, such as cGAMP, cyclic di-AMP, and cyclic di-GMP), or a derivative or combination thereof. In some embodiments, the immunomodulator comprises PAPC, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21, stimulon, vadimezan, AsA404 (DMXAA), a STING agonist (e.g., cyclic dinucleotides, such as cGAMP, cyclic di-AMP, and cyclic di-GMP), or a derivative or combination thereof. In some embodiments, the STING agonist is cyclic dinucleotide such as cGAMP.

In some embodiments, the immunomodulator is encapsulated in a liposome. In some embodiments, the immunomodulator is conjugated with a lipophilic moiety. In some embodiments, the lipophilic moiety is DOPE or cholesterol.

In another aspect, the invention provides a method of eliciting secretion of cytokines from immune cells in a mammalian subject comprising:

(a) administering a sonosensitizer to the subject,

(b) administering an immunomodulator to the subject,

(c) thereafter, exposing the subject to ultrasound radiation.

In some embodiments, the sonosensitizer comprises a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis (dithiolene) complex, bis (benzene-dithiolate) complex, iodoaniline dye, bis (S,O-dithiolene) complex, or a derivative or combination thereof.

In some embodiments, the sonosensitizer is encapsulated in a liposome. In some embodiments, the sonosensitizer is conjugated with a lipophilic moiety. In some embodiments, the lipophilic moiety is DOPE or cholesterol.

In some embodiments, the immunomodulator comprises LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist (e.g., cyclic dinucleotides, such as cGAMP, cyclic di-AMP, and cyclic di-GMP), or a derivative or combination thereof. In some embodiments, the STING agonist is cyclic dinucleotide such as cGAMP.

In some embodiments, the immunomodulator is encapsulated in a liposome. In some embodiments, the immunomodulator is conjugated with a lipophilic moiety. In some embodiments, the lipophilic moiety is DOPE or cholesterol.

In yet another aspect, the invention provides a method of promoting T cell activation in a mammalian subject comprising:

(a) administering a sonosensitizer to the subject,

(b) administering an immunomodulator to the subject, and

(c) thereafter, exposing the subject to ultrasound radiation.

In some embodiments, the sonosensitizer comprises a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis (dithiolene) complex, bis (benzene-dithiolate) complex, iodoaniline dye, or bis (S, O-dithiolene) complex, or a derivative or combination thereof.

In some embodiments, the sonosensitizer is encapsulated in a liposome. In some embodiments, the sonosensitizer is conjugated with a lipophilic moiety. In some embodiments, the lipophilic moiety is DOPE or cholesterol.

In some embodiments, the immunomodulator comprises LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21, stimulon, vadimezan, AsA404 (DMXAA), a STING agonist (e.g., a cyclic dinucleotide, such as cGAMP, cyclic di-AMP, and cyclic di-GMP), or a derivative or combination thereof. In some embodiments, the immunomodulator comprises CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist (e.g., cyclic dinucleotides, such as cGAMP, cyclic di-AMP, and cyclic di-GMP), or a derivative or combination thereof. In some embodiments, the STING agonist is cyclic dinucleotide such as cGAMP.

In some embodiments, the immunomodulator is encapsulated in a liposome. In some embodiments, the immunomodulator is conjugated with a lipophilic moiety. In some embodiments, the lipophilic moiety is DOPE or cholesterol.

A method of treating a tumor in a mammalian subject comprising:

(a) administering a sonosensitizer to the subject,

(b) administering an immunomodulator to the subject, and

(c) thereafter, exposing the tumor to ultrasound radiation.

In some embodiments, the sonosensitizer comprises a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis (dithiolene) complex, bis (benzene-dithiolate) complex, iodoaniline dye, bis (S,O-dithiolene) complex, or a derivative or combination thereof.

In some embodiments, the sonosensitizer is encapsulated in a liposome. In some embodiments, the sonosensitizer is conjugated with a lipophilic moiety.

In some embodiments, the lipophilic moiety is DOPE or cholesterol.

In some embodiments, the immunomodulator is selected from the group consisting of CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), STING agonists (e.g., cyclic dinucleotides, such as cGAMP, cyclic di-AMP, and cyclic di-GMP), and derivatives and combinations thereof. In some embodiments, the STING agonist is cyclic dinucleotide such as cGAMP.

In some embodiments, the immunomodulator is encapsulated in a liposome. In some embodiments, the immunomodulator is conjugated with a lipophilic moiety. In some embodiments, the lipophilic moiety is DOPE or cholesterol. In some embodiments, the mammalian subject is a human.

In still another aspect, the invention provides a kit for inducing secretion of cytokines that promote T cell activation in mammals, the kit comprising:

(a) a sonosensitizer comprising a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis (dithiolene) complex, bis (benzene-dithiolate) complex, iodoaniline dye, bis (S,O-dithiolene) complex, or a derivative or combination thereof; and

(b) an immunomodulator comprises LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist (e.g., cyclic dinucleotides, such as cGAMP, cyclic di-AMP, and cyclic di-GMP), or a derivative or combination thereof. In some embodiments, the STING agonist is cyclic dinucleotide such as cGAMP. In some embodiments, the immunomodulator comprises CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21, stimulon, vadimezan, AsA404 (DMXAA), a STING agonist (e.g., a cyclic dinucleotide, such as cGAMP, cyclic di-AMP, and cyclic di-GMP), or a derivative or combination thereof.

In some embodiments, the sonosensitizer or the immunomodulator is encapsulated in a liposome. In some embodiments, the sonosensitizer and the immune-modulator are both encapsulated in the same or in different liposomes.

In some embodiments, the sonosensitizer, the immunomodulator, or both are conjugated with one or more lipophilic moieties.

In some embodiments, the lipophilic moieties are selected from DOPE or cholesterol.

In a further aspect, the invention provides a pharmaceutical composition for parenteral administration to a subject comprising:

(a) a sonosensitizer comprising a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis (dithiolene) complex, bis (benzene-dithiolate) complex, iodoaniline dye, bis (S,O-dithiolene) complex, or a derivative or combination thereof;

(b) an immunomodulator comprising LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist (e.g., cyclic dinucleotides, such as cGAMP, cyclic di-AMP, and cyclic di-GMP), or a derivative or combination thereof; and

(c) a pharmaceutically acceptable carrier.

In some embodiments, the immunomodulator comprises CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21, stimulon, vadimezan, AsA404 (DMXAA), STING agonists (e.g., cyclic dinucleotides, such as cGAMP, cyclic di-AMP, and cyclic di-GMP), or a derivative or combination thereof. In some embodiments, the STING agonist is a cyclic dinucleotide, such as cGAMP. In some embodiments, the sonosensitizer or the immunomodulator is encapsulated in a liposome. In some embodiments, the sonosensitizer and the immunomodulator are both encapsulated in the same or in different liposomes.

In some embodiments, the sonosensitizer, the immunomodulator, or both are conjugated with one or more lipophilic moieties. In some embodiments, the lipophilic moieties are selected from DOPE and cholesterol.

In the embodiments described herein, inducing, eliciting or promoting a response means increasing a response. In some embodiments, the increase may be from 2-fold to 2000-fold or greater, or from any of 2, 5, 10, 20, 40 or 80-fold to any of 100, 200, 400, 800, 1600, or 3,200-fold.

In the embodiments described herein, ultrasound radiation refers to therapeutic ultrasound.

### Definitions

“Inducing” a response, such as inducing cytokine secretion, includes eliciting and/or enhancing or promoting a response. One of skill in the art readily understands that this is generally as compared to conditions that are otherwise the same except for a parameter of interest, or as compared to another condition (e.g., inducing cytokine secretion as a result of treatment with a sonosensitizer, an immunomodulator and ultrasound, as compared to no treatment or treatment with only one or two of a sonosensitizer, an immunomodulator and ultrasound). For example, “inducing” a response means increasing a response.

The term “pharmaceutically acceptable carrier,” as used herein, means one or more compatible solid or liquid filler, diluents or encapsulating substances which are suitable for administration into a human or another mammal.

“Pharmaceutically acceptable” means a non-toxic material that does not interfere with the effectiveness of the biological activity of the active ingredients. Pharmaceutically acceptable further means a non-toxic material that is compatible with a biological system such as a cell, cell culture, tissue, or organism.

“Carrier” denotes an organic or inorganic ingredient, natural or synthetic, with which the active ingredient is combined to facilitate the application. The characteristics of the carrier will depend on the route of administration. The components of the pharmaceutical compositions also are capable of being commingled with the sonosensitizers or the immunomodulators of the invention, and with each other, in a manner such that there is no interaction which would substantially impair the desired pharmaceutical efficacy. The pharmaceutically acceptable carrier must be sterile for in vivo administration. Physiologically and pharmaceutically acceptable carriers include diluents, fillers, salts, buffers, stabilizers, solubilizers, and other materials which are well known in the art.

“Parenteral” includes subcutaneous, intravenous, intramuscular, or infusion. It is preferred that intravenous or intramuscular routes are not used for long-term therapy and prophylaxis. Intravenous or intramuscular route of administration could, however, be preferred in emergency situations. Oral administration will be preferred for prophylactic treatment because of the convenience to the patient as well as the dosing schedule. It will be understood that the route of administration may also depend in some instances on the condition being treated. For example, if the condition is topical (e.g., atopic dermatitis or eczema), then the antagonists may be applied topically, intradermally or subcutaneously. Topical administration may be achieved using pads, gauzes, bandages, compression garments, creams, lotions, sprays, emollients, and the like, all of which comprise the antagonist of interest.

A “subject” refers to any mammal susceptible to having or having a tumor or otherwise in need of inducing the secretion of IL-1β, eliciting secretion of cytokines from immune cells or promoting T cell activation. The subjects may be human and non-human subjects.

### BRIEF DESCRIPTION OF THE DRAWINGS

FIGS. 1A and 1B show that ultrasound (US) can allow for precise ablation of tumors and activation of antitumor immunity. Sonosensitizers, upon exposure to ultrasound, can generate ROS, which not only has direct tumor killing effect in the ultrasound treated region, but also can synergize with immunomodulators to active the innate immune cells and induce secretion of critical cytokines, which, together with the antigens provided by dying tumor cells can induce activation of adaptive immune responses that can potentially eliminate all tumor cells. FIG. 1A shows a general approach, and FIG. 1B focuses on an approach utilizing liposomes.

FIG. 2 shows efficient ROS generation using ultrasound and sonosensitizer. Shown are ROS levels for indicated formulations in the absence or presence of ultrasound.

FIG. 3A-3B show activation of macrophages is composition and ultrasound dependent. FIG. 3A shows secretion of TNF-α from macrophages (RAW 264.7) after treatment with indicated formulations in the absence or presence of ultrasound (US). A=R848, B=ICG, US=ultrasound, and FIG. 3B shows the effect of ultrasound time on the secretion of TNF-α from macrophages (RAW 264.7).

FIG. 4A-FIG. 4B show IL-1β secretion is highly dependent on the composition and ultrasound. FIG. 4A shows secretion of IL-1 from iBMDM after treatment with indicated formulations in the absence or presence of ultrasound (US). A=PAPC, B=ICG, US=ultrasound. FIG. 4B shows the effect of ultrasound time on the secretion of IL-1β from macrophages iBMDM.

FIG. 5A-FIG. 5B show depletion of ROS compromised the cytokine secretion. FIG. 5A shows secretion of IL-1β from iBMDM after treatment with indicated formulations containing different concentrations (L=low concentration, M=medium concentration, H=High concentration) of ROS scavenger N-acetyl cysteine (NAC). FIG. 5B shows the viability of iBMDM after treatment with indicated formulations. The viability was measured using Trypan blue staining to exclude the interference of NAC on XTT assay.

FIG. 6A-FIG. 6B show increased ROS and macrophage activation both contribute to tumor killing in vitro. FIG. 6A shows the schematic of the coculture assay using the Transwell system. FIG. 6B shows the viability of tumor cells (CT26) cocultured with macrophages (RAW264.7) after treatment with indicated formulations in the absence or presence of ultrasound. A=R848, B=ICG, US=ultrasound.

FIG. 7 shows the schematic for the preparation of liposomes.

FIG. 8A-FIG. 8D show preparation and characterization of liposomes. Shown are the loading efficiency and size distribution lipo-R848/ICG (FIG. 8A-FIG. 8B), or lipo-PAPC/ICG (FIG. 8C-FIG. 8D).

FIG. 9 shows liposomes can significantly prolong the circulation time of the sonosensitizer ICG. Shown are the pharmacokinetic profiles of free ICG and liposomes containing ICG (Lipo-ICG) after intravenous injection in mice.

FIG. 10A-FIG. 10B show codelivery of sonosensitizer/immunomodulator using liposomes followed by ultrasound showed potent therapeutic effect. Balb/c mice were subcutaneously inoculated with 2×10⁵ CT26 cells/mouse on the right flank on day 0, and intravenously injected with formulations containing R848 and ICG (FIG. 10A) or PAPC and ICG (FIG. 10B) on day 10. Ultrasound (frequency: 1 MHz; duty cycle: 50%; power: 2 W/cm²) was applied for indicated groups of animals on day 11. Shown are the tumor growth curves after treatment.

FIG. 10C is a series of fluorescence images of tumor-bearing mice over time following intravenous injection of lipo-ICG or ICG.

FIG. 10D is a scatter plot quantifying the fluorescence in the mouse tumors from FIG. 10C. The data show mean±standard deviation from a representative experiment (n=3).

FIG. 11A is a series of FACS dot-plots showing intratumoral T cell responses in Balb/c mice on day 17. The mice were subcutaneously inoculated with 2×10⁵ CT26 cells/mouse on the right flank on day 0, and intravenously injected with formulations containing 60 ug/dose PAPC and 60 ug/dose ICG on day 10. Ultrasound (frequency: 1 MHz; duty cycle: 50%; power: 2.5 W/cm²) was applied one day after injection of indicated formulations.

FIG. 11B is a box plot showing percentage of CD8+ cells that are in the tumor of mice described in FIG. 11A. Whiskers, 5^{th} to 95^{th} percentile; n=8 for no treatment and n=9 for the other two groups. * p<0.05 analyzed by one-way ANOVA with Tukey's multiple comparisons post-test.

FIG. 11C is a box plot showing CD8/CD4 ratios for mice described in FIG. 11A. Whiskers, 5^{th} to 95^{th} percentile; n=8 for no treatment and n=9 for the other two groups. * p<0.05 analyzed by one-way ANOVA with Tukey's multiple comparisons post-test.

FIG. 11D is a scatter plot showing tumor volume growth over time in the mice described in FIG. 11A.

FIG. 11E is a plot showing Kaplan-Meier curves for the mice described in FIG. 11A.

FIG. 11F is a plot showing tumor volume growth over time in the Balb/c mice (with primary tumor regressed) that were rechallenged with 2×10⁵ CT26 cells/mouse on day 40 and observed for another 40 days. Shown are the individual CT26 tumor growth curves and animal survival (n=3)

FIG. 11G is a plot showing Kaplan-Meier curves for the mice described in FIG. 11F.

### DETAILED DESCRIPTION

In general, the invention provides compositions and methods useful in inducing secretion of a cytokine (e.g., IL-1β) from an immune cell (e.g., a professional antigen presenting cell, such as a macrophage), promoting T cell activation, or treating a tumor in a subject. The methods disclosed herein typically involve: (a) administering a sonosensitizer to the subject, (b) administering an immunomodulator to the subject, and (c) thereafter, exposing the subject to ultrasound radiation. The sonosensitizer and immunomodulator may be administered separately or concurrently. When administered concurrently, the immunomodulator and sonosensitizer may be administered in the same pharmaceutical composition. Alternatively, when administered concurrently, the immunomodulator and sonosensitizer may be administered in separate pharmaceutical compositions.

The pharmaceutical composition described herein may be liposomal formulations. Liposomes are typically formulated using lipids. The lipid for liposomal pharmaceutical composition may include, e.g., egg phosphatidylcholine (PC), egg phosphatidylglycerol (PG), soybean phosphatidylcholine (PC), hydrogenated soybean PC (HSPC), soybean phosphatidylglycerol (PG), brain phosphatidylserine (PS), brain sphingomyelin (SM), didecanoylphosphatidylcholine (DDPC), dierucoylphosphatidylcholine (DEPC), dimyristoylphosphatidylcholine (DMPC), distearoylphosphatidylcholine (DSPC), dilaurylphosphatidylcholine (DLPC), palmitoyloleoylphosphatidylcholine (POPC), palmitoylmyristoylphosphatidylcholine (PMPC), palmitoylstearoylphosphatidyl choline (PSPC), dioleoylphosphatidylcholine (DOPC), dioleoylphosphatidylethanolamine (DOPE), dilauroylphosphatidylglycerol (DLPG), distearoylphosphatidylglycerol (DSPG), dimyristoylphosphatidylglycerol (DMPG), dipalmitoylphosphatidylglycerol (DPPG), distearoylphosphatidylglycerol (DSPG), dioleoylphosphatidylglycerol (DOPG), palmitoyloleoylphosphatidylglycerol (POPG), dimyristoylphosphatidicacid (DMPA), dipalmitoylphosphatidic acid (DPPA), distearoylphosphatidic acid (DSPA), dimyristoylphosphatidylethanolamine (DMPE), dipalmitoylphosphatidylethanolamine (DPPE), dimyristoylphosphatidylserine (DMPS), dipalmitoylphosphatidylserine (DPPS), distearoylphosphatidylethanolamine (DSPE), dioleoylphosphatidylethanolamine (DOPE), dioleoylphosphatidylserine (DOPS), dipalmitoylsphingomyelin (DPSM), distearoylsphingomyelin (DSSM), or a combination thereof. Principles known for formulating compositions including immunomodulators can be leveraged in preparation of the pharmaceutical compositions and in the methods using the pharmaceutical compositions. Such principles can be found, e.g., in US 2018/0318414, the disclosure of which is incorporated by reference herein in its entirety.

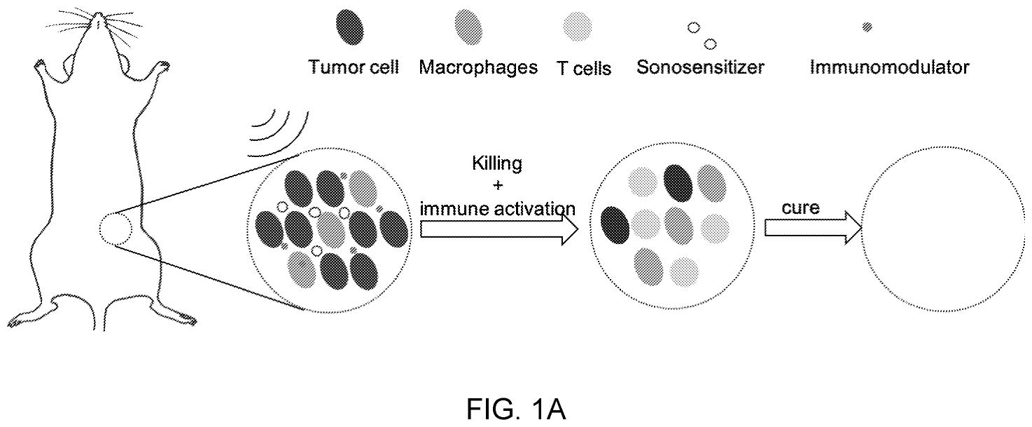

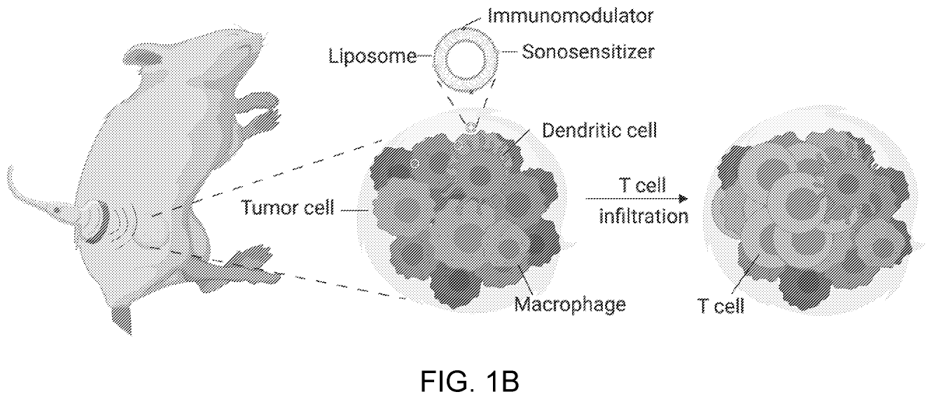

Here we show that the reactive oxygen species (ROS) generated with sonosensitizers and ultrasound can not only be used to kill tumor cells directly, but also can act as a switch to induce hallmarks of phagocyte hyperactivation, such as the secretion of IL-1β in the presence of 1-palmitoyl-2-arachidonoyl-sn-glycero-3-phosphocholine (PAPC). Moreover, removing any component from the combination (PAPC/sonosensitizer/ultrasound) completely abrogates the secretion of IL-1β from macrophages, and the activation can be easily tuned by changing the ultrasound parameters such as ultrasound exposure time. This approach enables precise control of the location and extent of immune cell activation and secretion of cytokines, which together with the antigens from dying tumor cells, can result in activation of adaptive immune responses (FIGS. 1A and 1B). To further improve the pharmacokinetic profiles of sonosensitizers and immunomodulators for in vivo applications, we pack these molecules in liposomes, which have a track record of good safety and can be easily manufactured under cGMP conditions. Our results indicate that injection of liposomes containing sonosensitizers such as ICG and immunomodulators such as PAPC followed by ultrasound can potently inhibit tumor growth compared with the injection of free drugs followed by ultrasound. These in vitro and in vivo results imply that sonosensitizers and ultrasound not only have a direct effect on the growth of tumor cells, but also can serve as a general platform to control the secretion of cytokines that are important for activation of T cells. Because this platform doesn't require identification of antigens, we envision it can be used to promote activation of T cells for multiple types of cancers.

In one aspect, the invention includes methods of inducing secretion of IL-1β, methods of eliciting secretion of cytokines from immune cells, methods of promoting T cell activation, and methods of treating a tumor in a mammalian subject. These methods include (a) administering a sonosensitizer to the subject, (b) administering an immunomodulator to the subject, and (c) thereafter, exposing the subject to ultrasound radiation.

In another aspect, the invention includes a kit for inducing secretion of IL-1β, a kit for eliciting secretion of cytokines from immune cells, a kit for promoting T cell activation, and a kit for treating tumors in mammals, the kit a sonosensitizer and an immunomodulator.

In yet another aspect, the invention includes pharmaceutical compositions for inducing secretion of IL-1β, eliciting secretion of cytokines from immune cells, promoting T cell activation, and/or treating tumors in mammals.

In any one or more of these aspects, the sonosensitizer may comprise a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular and intermolecular charge-transfer dye and dye complex, tropone, tetrazine, bis (dithiolene) complex, bis (benzene-dithiolate) complex, iodoaniline dye, bis (S, O-dithiolene) complex, or a combination thereof. It may be encapsulated in a liposome and/or conjugated with a lipophilic moiety, for example DOPE or cholesterol.

In any one or more of these aspects, the immunomodulator may comprise LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21, stimulon, vadimezan, AsA404 (DMXAA), a STING agonist (e.g., a cyclic dinucleotide, such as cGAMP, cyclic di-AMP, and cyclic di-GMP), or a derivative or combination thereof. In any one or more of these aspects, the immunomodulator may comprise CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist (e.g., a cyclic dinucleotide, such as cGAMP, cyclic di-AMP, and cyclic di-GMP), or a derivative or combination thereof. It may be encapsulated in a liposome and/or conjugated with a lipophilic moiety, for example DOPE or cholesterol.

The sonosensitizers and/or immunomodulators are administered to the subject in a therapeutically effective amount. A therapeutically effective amount is a dosage of the sonosensitizer and/or immunomodulator that is sufficient to provide a medically desirable result. In the methods of the invention, the therapeutically effective amount of the sonosensitizer and/or the immunomodulator may be that amount that is sufficient to elicit secretion of cytokines from immune cells, promote T cell activation, induce secretion of IL-1β, and/or induce or promote tumor regression.

The pharmaceutical compositions for inducing secretion of IL-1β, eliciting secretion of cytokines from immune cells, promoting T cell activation and/or treating tumors in a subject include a pharmaceutically acceptable carrier and a sonosensitizer and/or an immumodulator, either alone or in combination. The pharmaceutical preparations, as described above, are administered in effective amounts. For therapeutic applications, it is generally that amount sufficient to achieve a medically desirable result. In general, a therapeutically effective amount is that amount necessary to delay the onset of, inhibit the progression of, or halt altogether the particular condition being treated, for example cancer. As an example, the effective amount is generally that amount which serves to alleviate the symptoms (e.g., tumor growth etc.) of the disorders described herein. The effective amount will depend upon the mode of administration, the condition being treated and the desired outcome. It will also depend upon the stage of the condition, the severity of the condition, the age and physical condition of the subject being treated, the nature of concurrent therapy, if any, the duration of the treatment, the specific route of administration and like factors within the knowledge and expertise of the medical practitioner. For prophylactic applications, it is that amount sufficient to delay the onset of, inhibit the progression of, or halt altogether the condition being prevented, and may be measured by the amount required to prevent the onset of symptoms. Generally, doses of active compounds of the present invention would be from about 0.1 mg/kg per day to 1000 mg/kg per day, preferably from about 0.1 mg/kg to 200 mg/kg and most preferably from about 0.2 mg/kg to about 20 mg/kg, in one or more dose administrations daily, for one or more days. It is expected that doses ranging from 1-500 mg/kg, and preferably doses ranging from 1-100 mg/kg, and even more preferably doses ranging from 1-50 mg/kg, will be suitable. The preferred amount can be determined by one of ordinary skill in the art in accordance with standard practice for determining optimum dosage levels of the agent. It is generally preferred that a maximum dose of a sonosensitizer and/or immunomodulator that is the highest safe dose according to sound medical judgment be used. See Nair and Jacob, J Basic Clin Pharm 7(2): 27-31 (2016).

The sonosensitizers and/or immunomodulators may be administered alone or as part of one or more pharmaceutical compositions. Such pharmaceutical compositions may include the sonosensitizer and/or the immunomodulator in combination with any standard physiologically and/or pharmaceutically acceptable carriers that are known in the art. The compositions should be sterile and contain a therapeutically effective amount of the sonosensitizer and/or the immunomodulator in a unit of weight or volume suitable for administration to a subject.

Compositions suitable for parenteral administration comprise a sterile aqueous preparation of the sonosensitizer and/or immunomodulator that is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1,3-butane diol.

Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulations suitable for oral, subcutaneous, intravenous, intramuscular, etc. administrations can be found in Remington's Pharmaceutical Sciences, Mack Publishing Co., Easton, Pa.

A variety of administration routes are available. The mode selected will depend upon the drug selected, the severity of the condition being treated, and the dosage required for therapeutic efficacy. The methods of the invention may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes.

The pharmaceutical compositions may conveniently be presented in unit dosage form and may be prepared by any of the methods well-known in the art of pharmacy. All methods include the step of bringing the sonosensitizer and/or immunomodulator into association with a carrier that constitutes one or more accessory ingredients. In general, the compositions are prepared by uniformly and intimately bringing the sonosensitizer and/or immunomodulator into association with a liquid carrier, a finely divided solid carrier, or both, and then, if necessary, shaping the product.

Compositions suitable for oral administration may be presented as discrete units, such as capsules, tablets, lozenges, each containing a predetermined amount of the sonosensitizer and/or immunomodulator. Other compositions include suspensions in aqueous liquids or non-aqueous liquids such as a syrup, elixir or an emulsion.

The following are various exemplary compositions and methods which describe the invention. It is understood that other embodiments may be practiced given the general description provided above.

### EXAMPLES

### Methods

ROS Generation with Sonosensitizer and Ultrasound

The ROS was generated by exposing sonosensitizers such as ICG to ultrasound using ultrasound applicators. Exemplary ultrasound applicators and generator systems that can be used in conjunction with the embodiments herein disclosed include Mettler Electronics Sonicator™ series ultrasound devices (e.g. Sonicator™ 715, 716, 740, 740x), Mettler Electronics Sonicators Plus™ series ultrasound devices (e.g. Sonicator Plus™ 930, 940, 992, and 994), US Pro 2000™ portable ultrasound device, Chattanooga Inetlect TransPort™ ultrasound units. Other appropriate ultrasound applicators that may be chosen for use are within the level of skill in the art.

ROS levels were detected using a non-fluorescent probe that becomes fluorescent upon oxidation with ROS. Briefly, 0.5 mL of 1 mM DCFH-DA (Sigma, MO, USA) in ethanol was pretreated with 2 mL of 0.01 N NaOH (Fisher Scientific, NH, USA) and allowed to sit in the dark at room temperature for 30 min. The hydrolysate was then neutralized with 10 mL of 25 mM sodium phosphate buffer (Fisher Scientific, NH, USA) and kept on ice until use. PAPC and/or indocyanine green (ICG) in the final concentration of 10 ng/mL and 20 ng/mL, respectively, were added to the activated DCFH solution and ultrasound irradiation (frequency: 1 MHz; duty cycle: 50%; power: 2 W/cm²) was performed for different lengths of time (up to 5 min). The fluorescence signal was assessed by plate reader Infinite 200 Pro (Tecan, Männedorf, SUI) under excitation at 488 nm and emission at 525 nm.

### Cytokine Release In Vitro

RAW 264.7 macrophages (ATCC, VA, USA) were seeded in a 96 well plate at a density of 20,000 cells per well. Cells were incubated with 10 ng/mL R848 (Sigma, MO, USA) and/or 20 ng/mL ICG (Sigma, MO, USA) for 24 h. Ultrasound irradiation (frequency: 1 MHz; duty cycle: 50%; power: 2 W/cm²) was applied to these cells for up to 5 min. TNFα secretion was analyzed by mouse TNFα DuoSet ELISA (R&D Systems, MN, USA) following the manufacturer's instructions. To measure IL-1β secretion, immortal bone marrow derived macrophages or iBMDM (BCH, MA, USA)) were seeded in a 96 well plate at a density of 20,000 cells per well. Cells were incubated with 10 ng/mL PAPC (Avanti, AL, USA) and/or 20 ng/mL ICG for 24 h. Ultrasound irradiation (frequency: 1 MHz; duty cycle: 50%; power: 2 W/cm²) was applied to these cells for up to 5 min. IL-1β secretion was analyzed by mouse IL-1β ELISA (Invitrogen, CA, USA) following the manufacturer's instructions. In some experiments, cells were also treated with 0.5, 1, or 2 mM of ROS scavenger N-acetyl-cysteine (Sigma, MO, USA) right before treatment with ultrasound.

### Co-Culture Assay

RAW 264.7 macrophages were seeded in a Transwell insert at a density of 200,000 cells per insert (Corning, N.Y., USA) and CT26 (ATCC, VA, USA) were seeded in the lower compartment at a density of 200,000 cells per well, which was separated by a porous membrane (well area: 0.3 cm², insert size: 6.5 mm). Cells were incubated with ng/mL R848 and/or 20 ng/mL ICG for 24 h. Ultrasound irradiation (frequency: 1 MHz; duty cycle: 50%; power: 2 W/cm²) was performed for 5 min. The viability of tumor cells after treatment with different formulations in the absence or presence of ultrasound was determined by XTT Cell Proliferation Assay Kit (ATCC, VA, USA) following the manufacturer's instructions.

### Preparation of Liposome Formulations

Proper amount of 1,2-dipalmitoyl-sn-glycero-3-phosphocholine (Avanti, AL, USA), 1,2-distearoyl-sn-glycero-3-phosphoethanolamine-N-[methoxy (polyethylene-glycol)-2000] (Avanti, AL, USA), and cholesterol (Sigma, MO, USA) were dissolved in 0.5 mL ethanol, which was slowly added to 5 mL aqueous buffer and incubated for 10 min at 60° C. The lipid suspension was extruded through the 100 nm polycarbonate membrane using the extruder (Avanti, AL, USA) to obtain blank liposomes. Ethanol was removed by dialysis overnight at 4° C.

Whilst the foregoing liposome was used in this experiment, other lipids may be employed. The lipid for liposome preparation may comprise egg phosphatidylcholine (PC), egg phosphatidylglycerol (PG), soybean phosphatidylcholine (PC), hydrogenated soybean PC (HSPC), soybean phosphatidylglycerol (PG), brain phosphatidylserine (PS), brain sphingomyelin (SM), didecanoylphosphatidylcholine (DDPC), dierucoylphosphatidylcholine (DEPC), dimyristoylphosphatidylcholine (DMPC), distearoylphosphatidylcholine (DSPC), dilaurylphosphatidylcholine (DLPC), palmitoyloleoylphosphatidylcholine (POPC), palmitoylmyristoylphosphatidylcholine (PMPC), palmitoylstearoylphosphatidyl choline (PSPC), dioleoylphosphatidylcholine (DOPC), dioleoylphosphatidylethanolamine (DOPE), dilauroylphosphatidylglycerol (DLPG), distearoylphosphatidylglycerol (DSPG), dimyristoylphosphatidylglycerol (DMPG), dipalmitoylphosphatidylglycerol (DPPG), distearoylphosphatidylglycerol (DSPG), dioleoylphosphatidylglycerol (DOPG), palmitoyloleoylphosphatidylglycerol (POPG), dimyristoylphosphatidicacid (DMPA), dipalmitoylphosphatidic acid (DPPA), distearoylphosphatidic acid (DSPA), dimyristoylphosphatidylethanolamine (DMPE), dipalmitoylphosphatidylethanolamine (DPPE), dimyristoylphosphatidylserine (DMPS), dipalmitoylphosphatidylserine (DPPS), distearoylphosphatidylethanolamine (DSPE), dioleoylphosphatidylethanolamine (DOPE), dioleoylphosphatidylserine (DOPS), dipalmitoylsphingomyelin (DPSM), distearoylsphingomyelin (DSSM), or a combination thereof.

Liposomes are commercially available from Gibco BRL, for example, as LIPOFECTIN™ and LIPOFECTACE™, which are formed of cationic lipids such as N-[1-(2, 3 dioleyloxy)-propyl]-N, N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, V. 3, p. 235-241 (1985).

To load the sonosensitizer in liposomes, ICG was firstly conjugated to a lipid tail such as DOPE before incubation with preformed blank liposomes at room temperature for 30 min. Unloaded ICG was removed by using the PD-10 column (GE Healthcare).

In some embodiments, the sonosensitizer for ROS generation comprises a porphyrin, cyanine (e.g., indocyanine green (ICG)), merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis (dithiolene) complex, bis (benzene-dithiolate) complex, iodoaniline dye, bis (S, O-dithiolene) complex, or a derivative or combination thereof. In some embodiments, the sonosensitizer is conjugated with a lipid tail (e.g. DOPE or cholesterol or any other lipophilic moieties) to improve the loading efficiency in liposomes.

To load PAPC (Avanti, AL, USA) in liposomes, the proper amount of PAPC in DMSO stock solution was incubated with liposomes containing ICG at room temperature for 30 min and the obtained formulation was used without further purification. To load R848 (Sigma, MO, USA) in liposomes, blank liposomes were firstly prepared in 250 mM ammonium sulfate, and the external ammonium sulfate was exchanged to 10% sucrose by dialysis overnight at 4° C., followed by incubation with R848 at 55° C. for 30 min and removal of unloaded R848 by dialysis overnight at 4° C.

In some embodiments, the immunomodulator comprises LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), STING, R848 (Resiquimod) PAPC, or a derivative or combination thereof. In some embodiments, the immunomodulator comprises CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), STING, R848 (Resiquimod) PAPC, or a derivative or combination thereof. In some embodiments, the immunomodulator is conjugated with a lipid tail (e.g., DOPE or cholesterol or any other lipophilic moieties) to improve the loading efficiency in liposomes.

### Characterization of Liposomes

To measure the size of liposomes, 10 ul of liposomes were diluted to 2 mL with PBS and the size was measured with Zeta sizer. To measure the amount of ICG, 10 ul liposomes were added with 190 ul DMSO and the fluorescence was measured at Ex=780 nm, Em=810 nm.

### Pharmacokinetic and Biodistribution Study

C57BL/6 mice were intravenously injected with free ICG or lipo-ICG (liposome encapsulated ICG). At predetermined time points (0.25, 1, 3, 7, and 24 h post injection), 50 ul blood were collected in Microvette 500 Z-gel tubes by submandibular bleeding and kept on ice. The samples were centrifuged at 10,000 g for 5 min at room temperature, and 10 ul of the serum were diluted to 100 ul with PBS and the fluorescence intensity was measured at Ex=780, Em=810 nm.

To investigate the biodistribution profile of lipo-ICG, animals were intravenously injected with 30 ug/dose of free ICG or lipo-ICG and the animals were imaged at indicated time points (3, 24, 48, and 72 h post injection) using the Xtreme fluorescence imaging system.

### Therapeutic Study

Balb/c mice were subcutaneously inoculated with 2×10⁵ CT26 cells/mouse on the right flank on day 0, and intravenously injected with indicated formulations on day 10. In some experiments, ultrasound (frequency: 1 MHz; duty cycle: 50%; power: 2-2.5 W/cm²) was applied for indicated groups of animals on day 11. In some experiments, animals in whom primary tumors were eliminated were rechallenged with the same tumor cells on the left flank on indicated days. The tumor volume was measured by 3 times/week and the volume was calculated with the following equation: volume=0.52×length×width². Animals were euthanized when the tumors reached 15 mm in diameter or had active ulceration.

### Statistical Analysis

All statistical analysis was performed with GraphPad Prism 7 (GraphPad, CA, USA). All data were analyzed with one-way or two-way ANOVA test to determine the statistical difference of means among various groups, followed by the recommended multiple comparisons tests. A p-value less than 0.05 was considered statistically significant.

### Results

ROS Generation with Sonosensitizer and Ultrasound

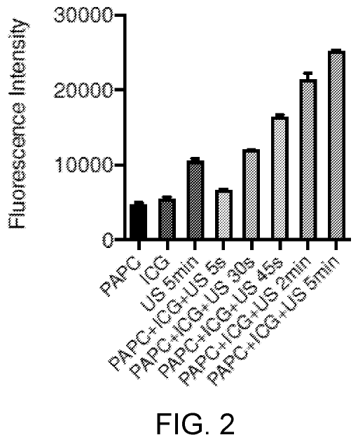

We first established the method to generate ROS using sonosensitizer ICG and ultrasound, which have well documented safety profiles in clinical settings. ROS levels were detected using a probe that became fluorescent upon oxidation with ROS. Immunomodulator or sonosensitizer alone induced background levels of ROS (FIG. 2). Interestingly, ultrasound alone induced slightly higher levels of ROS compared with immunomodulator or sonosensitizer alone. This is because molecules in the environment, including the probe used to detect ROS levels may absorb the energy of ultrasound and generate ROS when the excited electron returns to the ground state, thus leading to the oxidation of the ROS probe. Although ultrasound alone induced some ROS, the efficiency was significantly weaker than the combination of sonosensitizer and ultrasound, which induced over 2.5-fold more ROS under the same ultrasound condition. Moreover, the ROS generation was highly dependent on the ultrasound parameters such as ultrasound time, and higher levels of ROS were induced with longer ultrasound time. These results indicate the combination of sonosensitizer/ultrasound, but not sonosensitizer or ultrasound alone is a promising approach to generate ROS in a highly controllable manner for therapeutic applications.

### Cytokine Release

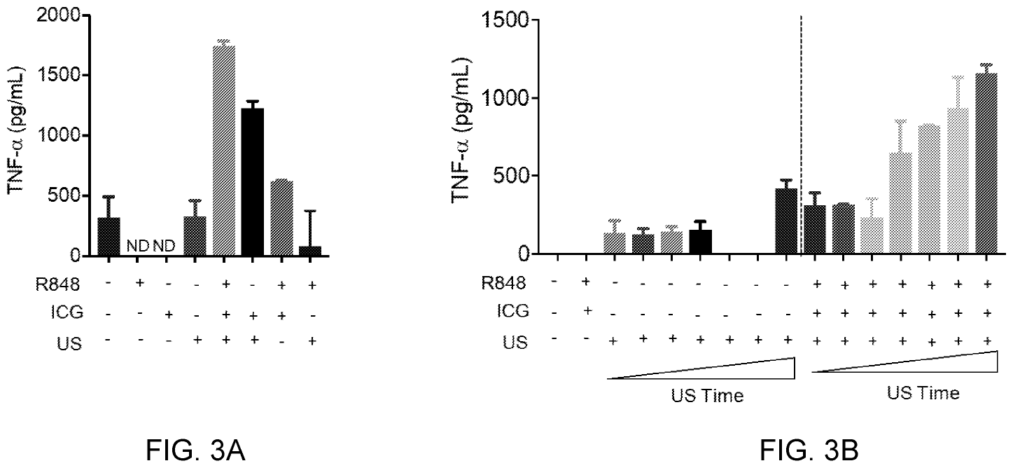

To learn whether the inducible ROS generated with ultrasound and sonosensitizer can be used to synergize with immunomodulators, we firstly measured the cytokine TNFα release from RAW264.7 macrophages after treating these cells with TLR7/8 agonist R848, ICG, ultrasound, or their combinations. R848, ICG, or ultrasound alone didn't induce significant TNFα release compared with the no treatment control (FIG. 3A). Surprisingly, when they were combined together, over 7-fold higher levels of TNFα were secreted from macrophages. Moreover, removing any component (R848, ICG, or ultrasound) from the combination significantly compromised the activation of macrophages, as shown by the decrease of TNFα release. We also found TNFα release was dependent on the ultrasound time, and higher TNFα levels were induced with longer ultrasound time (FIG. 3B).

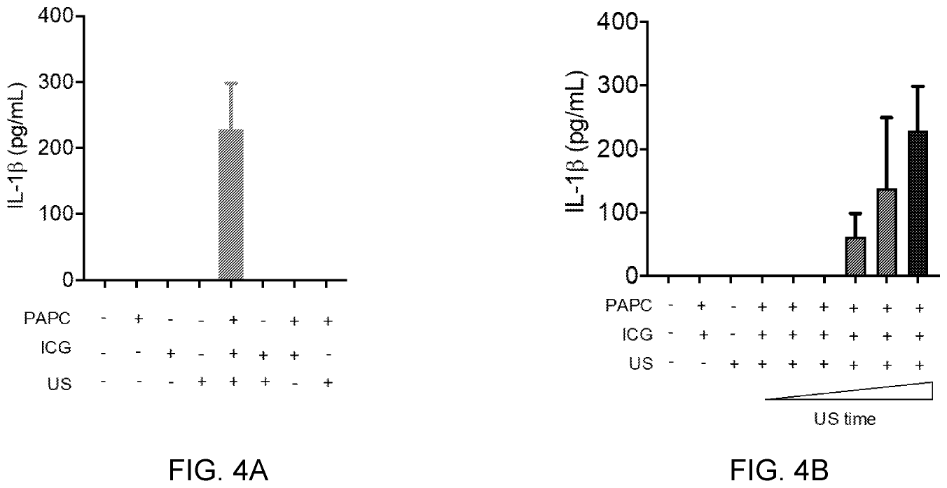

We also tested the effect of inducible ROS on other immunomodulators such as PAPC. The release of IL-1β from iBMDM was chosen as a marker to evaluate the activation of innate immune cells. PAPC, ICG, or ultrasound alone didn't induce any detectable level of IL-1β. Strikingly, combination of PAPC, ICG and ultrasound induced high levels of IL-1β and depletion of any component from the combination completely abrogated the release IL-1β (FIG. 4A). We also found IL-1β release was dependent on the ultrasound time, and higher IL-1β levels were induced with longer ultrasound time (FIG. 4B). All together, these results indicate the macrophage activation, as shown by the secretion of TNFα or IL-1, is highly dependent on the composition and can be tuned by changing ultrasound parameters.

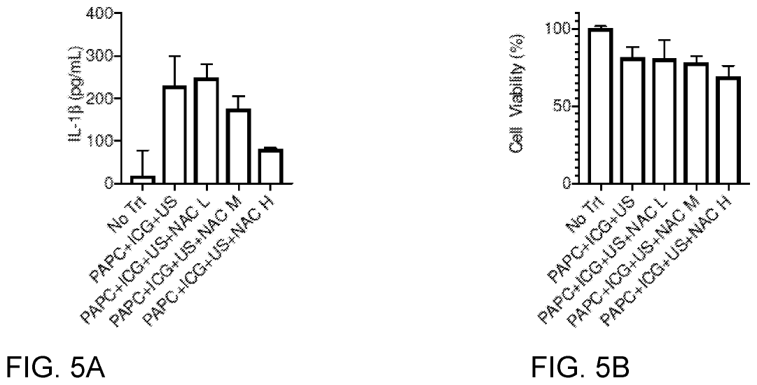

To confirm whether ROS was the major factor that triggers the activation of immune cells, we used ROS scavenger N-acetyl-cysteine (NAC) to deplete ROS from the group receiving the combination of PAPC+ICG+US. Depletion of ROS significantly compromised the activation of iBMDM, as shown by the NAC dose dependent reduction of IL-1β (FIG. 5A). To understand whether the reduction of IL-1β was due to the toxicity of ROS scavenger, we measured the viability of iBMDM receiving PAPC+ICG+US plus different concentrations of ROS scavenger. iBMDM had similar viabilities after treatment with PAPC+ICG+US in the absence or presence of ROS scavenger (FIG. 5B). These results indicated that inducible ROS generated with ultrasound was the major factor that synergizes with the immunomodulator to induce secretion of cytokines.

### In Vitro Tumor Cell Killing Effect

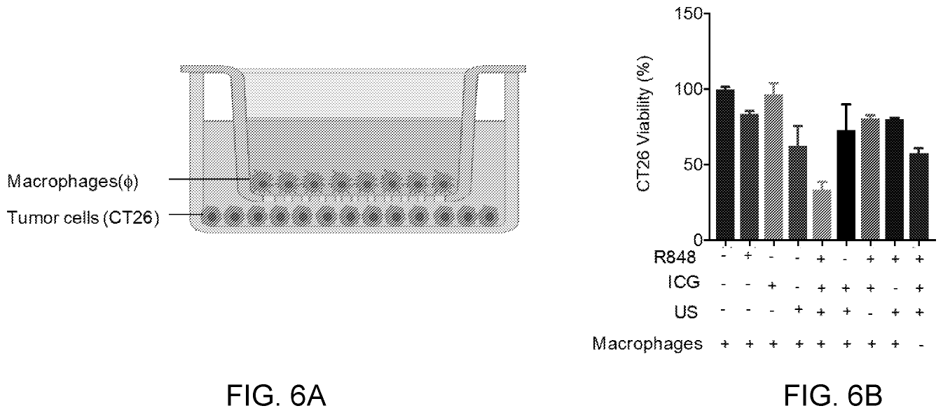

To evaluate the effect of different combinations on the viability of cancer cells, macrophages were cocultured with CT26 cancer cells using a transwell system (FIG. 6A), followed by treatment with indicated compositions. R848, ICG, or ultrasound alone only caused a modest decrease of CT26 cell viability, but the combination of all three components significantly decreased the viability to lower than 50%. Removing any component from the combination also significantly compromised the cancer cell killing. Interestingly, we found removing macrophages from the group receiving combination therapy also significantly compromised the tumor killing effect, indicating activation of macrophages can also contribute to kill cancer cells (FIG. 6B). This is not surprising as cytokines such as TNFα released from macrophages are known to have tumor killing effect.

### Preparation of Liposomes

Having shown the inducible ROS can synergize with immunomodulators and induce secretion of cytokines that are critical for adaptive immune responses, we sought to evaluate their potential for the treatment of tumors. However, sonosensitizers and immunomodulators are small molecules that can be rapidly eliminated in vivo and not be readily available simultaneously in the tissue of interest. This motivated us to improve the pharmacokinetic profiles and colocalization of sonosensitizers and immunomodulators. To achieve this, we chose to use liposomes, a type of lipid-based vesicles and had a track record of good safety and biocompatibility for in vivo delivery of sonosensitizers and immunomodulators.

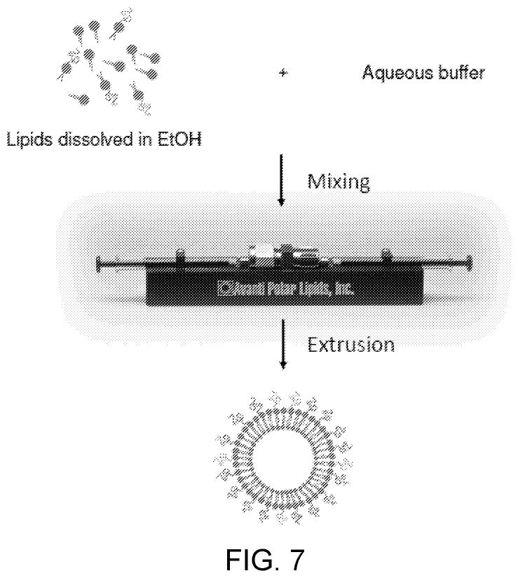

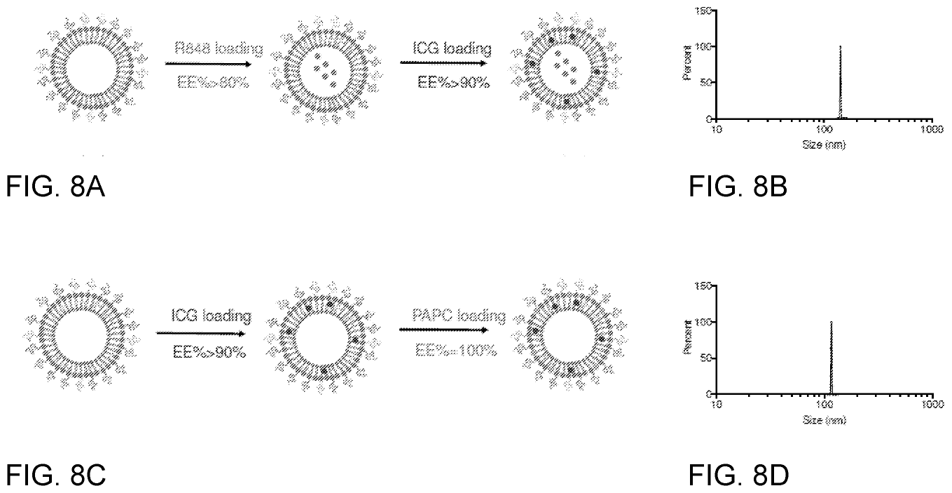

Blank liposomes were prepared by mixing the ethanol solution of lipids with selected aqueous phase at 60° C., followed by passing through the 100 nm polycarbonate membrane to generate homogeneous liposomes (FIG. 7). To prepare liposomes containing the immunomodulator R848 and sonosensitizer ICG (FIG. 8A and FIG. 8B), R848 was firstly loaded in liposomes using the active loading protocol, and the loading efficiency was over 80%, which was significantly higher than ˜10% achieved using the passive loading protocol. To efficiently load the sonosensitizer in liposomes, it was conjugated to a lipid tail before incubation with preformed liposomes at room temperature. The loading efficiency of lipid-conjugated sonosensitizer was over 95%, while the loading efficiency of lipid-free sonosensitizer was less than 20%. Moreover, because the sonosensitizer loading process was separated from the preparation of liposomes, which require a relatively high temperature (60° C.), we were able to protect the sonosensitizer from exposure to heat and minimize the loss of their activity. To prepare liposomes containing the immunomodulator PAPC and sonosensitizer ICG (FIG. 8C and FIG. 8D), lipid-conjugated ICG was firstly incubated with preformed liposomes, with a loading efficiency over 95%. Then PAPC was incubated with the obtained lipo-ICG to obtain lipo-PAPC/ICG.

### Pharmacokinetics and Biodistribution Study

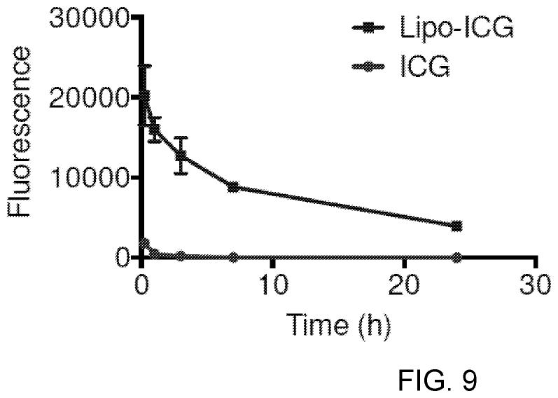

To investigate the pharmacokinetics of free drugs versus liposome formulations, C57BL/6 mice were intravenously injected with free ICG or lipo-ICG and the concentrations of ICG at different time points post injection were measured using the plate reader. Free ICG was not detectable within a few minutes after injection. In contrast, the liposomal ICG exhibited a significantly longer circulation time (FIG. 9) and significantly larger area under the curve (AUC).

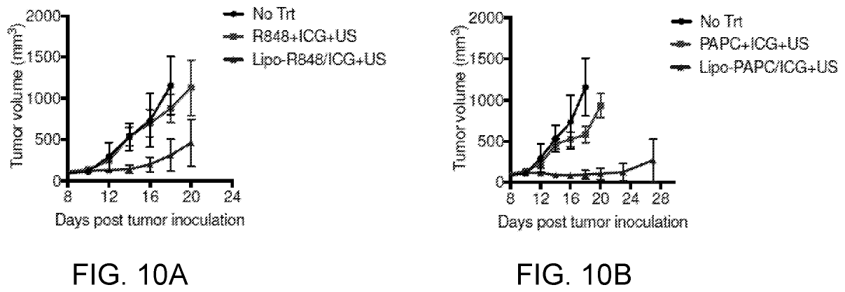

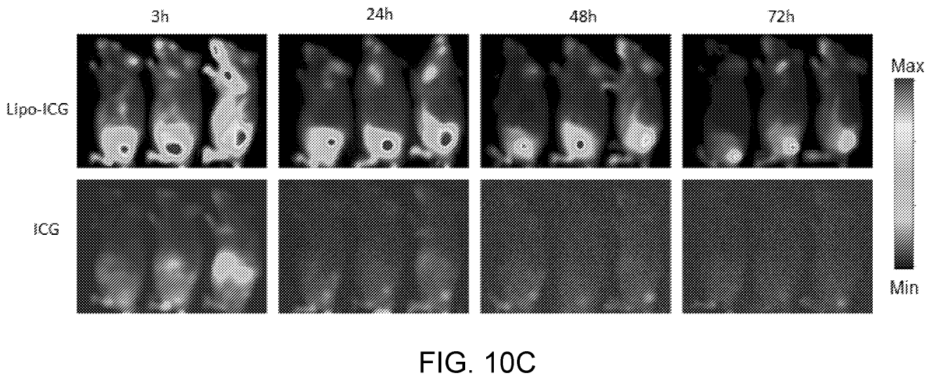

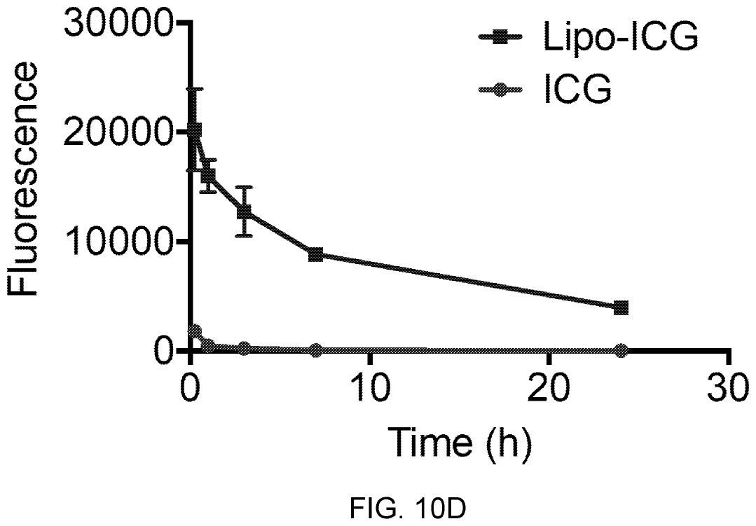

Biodistribution of ICG was assessed using fluorescence imaging over time following intravenous injection of ICG or lipo-ICG in the tumor-bearing mice (FIG. 10C). Quantification of the fluorescence in the tumor revealed superior competence of lipo-ICG at intra-tumoral delivery of ICG than non-liposomal formulation of ICG (FIG. 10D).

### Therapeutic Study

To evaluate the therapeutic efficacy, CT26 tumor-bearing mice were intravenously injected with the physical mixture of R848+ICG or liposomes containing R848 and ICG (lipo-R848/ICG) on day 10 post inoculation of tumor cells, followed by ultrasound treatment on day 11. While free R848+ICG+US only showed marginal tumor growth inhibition, lipo-R848/ICG had significantly better tumor growth inhibition compared with no treatment control and R848+ICG+US (FIG. 10A). Similarly, PAPC+ICG+US only had minimal effect on the tumor growth, but lipo-PAPC/ICG+US showed significantly better tumor growth inhibition compared with no treatment and PAPC+ICG+US (FIG. 10B).

Activating robust antitumor immune responses requires several signals, including tumor antigens, and activation of innate immune cells such as macrophages and dendritic cells, which can result in further activation of T cell responses. Our results indicate application of ultrasound with sonosensitizers co-packaged with agents that activate dendritic cells creates the basis for this synergistic signaling. In particular, ultrasound and sonosensitizer generated ROS can kill tumor cells and provide tumor antigens. ROS can also synergize the activation of innate immune cells such as macrophages and dendritic cells by immunomodulators, resulting in generation of critical cytokines that promote T cell activation. Because robust immune responses were induced only when all signals are present in the same place. The liposomes are really a way to ensure the signals are all in the same place. Remarkably, there is so little effect when the signals are not colocalized (administered free in the blood). This is also consistent with the in vitro finding that all three signals must be present. These results indicated the improved pharmacokinetic profiles and colocalized delivery of sonosensitizers and immunomodulators achieved by liposomes can potentiate the synergistic effect of immune activation and antitumor efficacy.

A dosing regimen may be optimized as needed by one of skill in the art. See for example, Nair and Jacob, J Basic Clin Pharm 7(2): 27-31 (2016). IVIS imaging may be used to monitor the biodistribution profiles of sonosensitizers in the free form and in the liposomal form at different time points. This will help identify the optimal time frame when ultrasound should be applied. The efficacy of ultrasound application on draining lymph nodes can be evaluated as an alternative or complementary target to potentiate the initiation of anti-tumor immunity. Liposomes containing the immune-modulator PAPC and sonosensitizer ICG reach the draining lymph nodes (dLN) upon i.v. injection. Thus, dLN serves as a complementary target for ultrasound application to boost the activation of myeloid cells, which could migrate to capture tumor antigens, and prime new tumor-specific CTLs. In addition, IL-1β cytokine secretion by macrophages and dendritic cells in the dLN upon ultrasound application could act as a licensing signal to enable optimal memory and effector T cells function. This process potentially broadens the range of antigens that are recognized, endowing the newly primed tumor-specific CTLs with optimal effector and memory capabilities and increasing the efficiency of anti-tumor immunity. Targeting the dLN with ultrasound is applied alternatively in many cases where solid tumors are located in deep tissues that cannot effectively be reached by ultrasound frequencies.

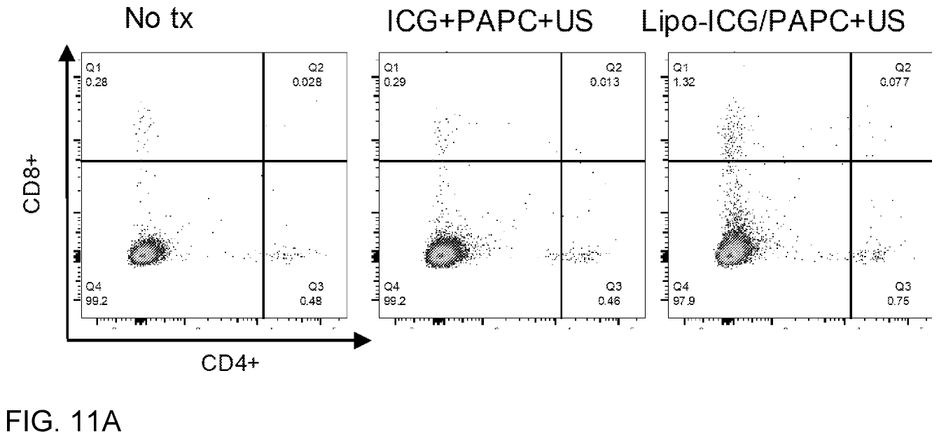

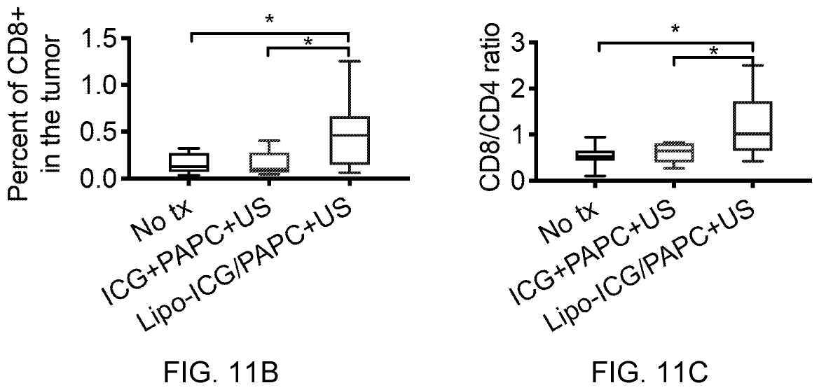

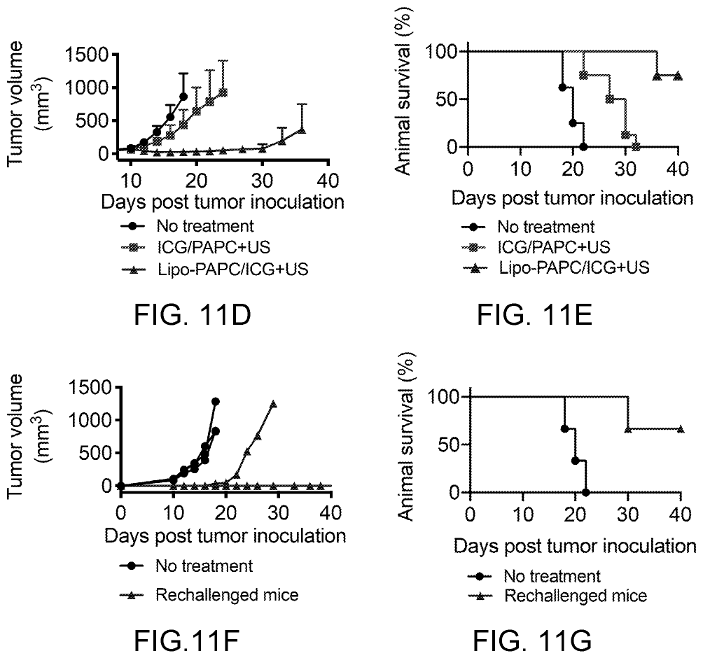

After treating tumor-bearing mice with different formulations+/−ultrasound, intratumoral immune responses were evaluated using flow cytometry (FIG. 11A). In particular, infiltration of CD8+ and CD4+ T cells were investigated after digesting the tumor tissue into single cell suspension (FIGS. 11B and 11C). The mice were monitored for 40 days for their tumor volumes and survival (FIGS. 11D and 11E). Those mice that had primary tumor regressed were rechallenged with 2×10⁵ CT26 cells/mouse on day 40 and observed for another 40 days (FIGS. 11F and 11G).

Additional models, such as breast cancer or melanoma, are available and can be used by the skilled artisan using known methods. Depending on the results, checkpoint inhibitors may be used in some experiments to show the synergy between our platform and ICB.

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references, scientific articles, patent publications, and any other documents cited herein are hereby incorporated by reference for the substance of their disclosure.

The invention is also described by the following enumerated embodiments.

1. A method of inducing cytokine secretion, comprising:

(a) contacting mammalian antigen presenting cells (APCs) with a sonosensitizer and an immunomodulator; and

(b) exposing the APCs of (a) to ultrasound radiation for a period of time sufficient to induce cytokine secretion by the APCs.

2. The method of embodiment 1, wherein the cytokine comprises one or both of IL-1β and TNF-α.

3. The method of embodiment 1 or embodiment 2, wherein the APCs comprise macrophages.

4. The method of any one of embodiments 1-3, wherein the APCs are present in a mammalian subject.

5. The method of embodiment 4, wherein the mammalian subject has a tumor and contacting and exposing the APCs results in killing cells of the tumor.

6. A method of inducing secretion of IL-1β in a mammalian subject comprising:

(a) administering a sonosensitizer to the subject,

(b) administering an immunomodulator to the subject, and

(c) thereafter, exposing the subject to ultrasound radiation.

7. The method according to any one of embodiments 1-6, wherein the sonosensitizer comprises a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular and intermolecular charge-transfer dye or dye complex, tropone, tetrazine, bis(dithiolene) complexe, bis (benzene-dithiolate) complexe, iodoaniline dye, bis (S,O-dithiolene) complex, or a combinations thereof, optionally, wherein the sonosensitizer comprises a cyanine.

8. The method according to any one of embodiments 1-7, wherein the sonosensitizer is encapsulated in a liposome.

9. The method according to any one of embodiments 1-8, wherein the sonosensitizer is conjugated with a lipophilic moiety.

10. The method according to embodiment 9, wherein the lipophilic moiety is DOPE or cholesterol.

11. The method according to any one of embodiments 1-10, wherein the immunomodulator comprises LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof, optionally wherein the immunomodulator comprises one or both of R848 and PAPC.

12. The method according to any one of embodiments 1-10, wherein the immunomodulator comprises CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof, optionally wherein the immunomodulator comprises one or both of R848 and PAPC.

13. The method according to any one of embodiments 1-12, wherein the immunomodulator is encapsulated in a liposome.

14. The method according to any one of embodiments 1-13, wherein the immunomodulator is conjugated with a lipophilic moiety.

15. The method according to embodiment 14, wherein the lipophilic moiety is DOPE or cholesterol.

16. A method of eliciting secretion of cytokines from immune cells in a mammalian subject comprising:

(a) administering a sonosensitizer to the subject,

(b) administering an immunomodulator to the subject,

(c) thereafter, exposing the subject to ultrasound radiation.

17. The method according to embodiment 15, wherein the sonosensitizer comprises a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular and intermolecular charge-transfer dye or dye complex, tropone, tetrazine, bis(dithiolene) complexe, bis (benzene-dithiolate) complexe, iodoaniline dye, bis (S,O-dithiolene) complex, or a combination thereof.

18. The method according to embodiment 16 or 17, wherein the sonosensitizer is encapsulated in a liposome.

19. The method according to any one of embodiments 16-18, wherein the sonosensitizer is conjugated with a lipophilic moiety.

20. The method according to embodiment 19, wherein the lipophilic moiety is DOPE or cholesterol.

21. The method according to any one of embodiments 16-20, wherein the immunomodulator comprises LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof.

22. The method according to any one of embodiments 16-20, wherein the immunomodulator comprises CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof.

23. The method according to any one of embodiments 16-22, wherein the immunomodulator is encapsulated in a liposome.

24. The method according to any one of embodiments 16-23, wherein the immunomodulator is conjugated with a lipophilic moiety.

25. The method according to embodiment 24, wherein the lipophilic moiety is DOPE or cholesterol.

26. A method of promoting T cell activation in a mammalian subject comprising:

(a) administering a sonosensitizer to the subject,

(b) administering an immunomodulator to the subject, and

(c) thereafter, exposing the subject to ultrasound radiation.

27. The method according to embodiment 26, wherein the sonosensitizer comprises a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis(dithiolene) complex, bis(benzene-dithiolate) complex, iodoaniline dye, and bis(S,O-dithiolene) complex, or a combination thereof.

28. The method according to embodiment 26 or 27, wherein the sonosensitizer is encapsulated in a liposome.

29. The method according to any one of embodiments 26-28, wherein the sonosensitizer is conjugated with a lipophilic moiety.

30. The method according to embodiment 29, wherein the lipophilic moiety is DOPE or cholesterol.

31. The method according to any one of embodiments 26-30, wherein the immunomodulator comprises LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof.

32. The method according to embodiment 31, wherein the immunomodulator comprises CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof.

33. The method according to any one of embodiments 26-32, wherein the immunomodulator is encapsulated in a liposome.

34. The method according to any one of embodiments 26-32, wherein the immunomodulator is conjugated with a lipophilic moiety.

35. The method according to embodiment 34, wherein the lipophilic moiety is DOPE or cholesterol.

36. A method of treating a tumor in a subject comprising:

(a) administering a sonosensitizer to the subject,

(b) administering an immunomodulator to the subject, and

(c) thereafter, exposing the tumor to ultrasound radiation.

37. The method according to embodiment 36, wherein the sonosensitizer comprises a porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis(dithiolene) complex, bis(benzene-dithiolate) complex, iodoaniline dye, or bis(S,O-dithiolene) complex, or a combination thereof.

38. The method according to embodiment 36 or 37, wherein the sonosensitizer is encapsulated in a liposome.

39. The method according to any one of embodiments 36-38, wherein the sonosensitizer is conjugated with a lipophilic moiety.

40. The method according to embodiment 39, wherein the lipophilic moiety is DOPE or cholesterol.

41. The method according to any one of embodiments 36-40, wherein the immunomodulator comprises LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof.

42. The method according to any one of embodiments 36-40, wherein the immunomodulator comprises CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof.

43. The method according to any one of embodiments 36-42, wherein the immunomodulator is encapsulated in a liposome.

44. The method according to any one of embodiments 36-43, wherein the immunomodulator is conjugated with a lipophilic moiety.

45. The method according to embodiment 44, wherein the lipophilic moiety is DOPE or cholesterol.

46. The method according to any one of embodiments 1-45, wherein mammalian cells are human cells and the mammalian subject is a human.

47. The method according to any one of embodiments, 1-46, wherein the immunomodulator comprises PAPC.

48. The method according to any one of embodiments 1-47, wherein the immunomodulator comprises R848.

49. The method according to any one of embodiments 1-48, wherein the sonosensitizer is a cyanine.

50. The method according to embodiment 49, wherein the sonosensitizer is indocyanine green.

51. A kit for inducing secretion of cytokines that promote T cell activation in mammals, the kit comprising:

(a) a sonosensitizer comprising porphyrin, cyanine, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis(dithiolene) complex, bis(benzene-dithiolate) complex, iodoaniline dye, and bis (S,O-dithiolene) complex, or a combination thereof; and

(b) an immunomodulator comprising LPS, MPL, R848, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof.

52. The kit according to embodiment 51, wherein the immunomodulator comprises CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), PAPC, Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof.

53. The kit according to embodiment 51 or 52, wherein either the sonosensitizer or the immunomodulator is encapsulated in a liposome.

54. The kit according to embodiment 53, wherein the sonosensitizer and the immunomodulator are both encapsulated in the same or in different liposomes.

55. The kit according to any one of embodiments 51-54, wherein the sonosensitizer, immunomodulator, or both are conjugated with one or more lipophilic moieties.

56. The kit according to embodiment 55, where the lipophilic moieties are selected from DOPE or cholesterol.

57. The kit according to any one of embodiments 51-56, wherein the immunomodulator comprises PAPC.

58. The kit according to any one of embodiments 51-57, wherein the immunomodulator comprises R848.

59. The kit according to any one of embodiments 51-58, wherein the sonosensitizer is a cyanine.

60. The kit according to embodiment 59, wherein the sonosensitizer is indocyanine green.

61. A pharmaceutical composition for parenteral administration to a subject comprising:

(a) a sonosensitizer comprising a cyanine, porphyrin, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis (dithiolene) complex, bis (benzene-dithiolate) complex, iodoaniline dye, bis (S, O-dithiolene) complex, or a combination thereof; and

(b) an immunomodulator comprising PAPC, R848, LPS, MPL, R837, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, resiquimod (R848), gardiquimod, 3M-052, SRL172, beta-glucan, Pam3Cys, Aquila's QS21 stimulon, vadimezan, AsA404 (DMXAA), a STING agonist, or a combination thereof; and

(c) a pharmaceutically acceptable carrier.

62. The pharmaceutical composition according to embodiment 61, wherein the immunomodulator comprises PAPC, resiquimod (R848), CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, gardiquimod, 3M-052, SRL172, beta-glucan, Pam3Cys, Aquila's QS21 stimulon, vadimezan, AsA404 (DMXAA), a STING agonist, or a combination thereof.

63. The pharmaceutical composition according to embodiment 61 or 62, wherein either the sonosensitizer or the immunomodulator is encapsulated in a liposome.

64. The pharmaceutical composition according to any one of embodiments 61-63, wherein the sonosensitizer and the immunomodulator are both encapsulated in the same or in different liposomes.

65. The pharmaceutical composition according to any one of embodiments 61-64, wherein either the sonosensitizer, immunomodulator, or both are conjugated with one or more lipophilic moieties.

66. The pharmaceutical composition according to embodiment 65, where the lipophilic moieties are selected from DOPE or cholesterol.

67. The pharmaceutical composition according to any one of embodiments 61-66, wherein the immunomodulator comprises PAPC.

68. The pharmaceutical composition according to any one of embodiments 61-67, wherein the immunomodulator comprises R848.

69. The pharmaceutical composition according to any one of embodiments 61-68, wherein the sonosensitizer is a cyanine.

70. The pharmaceutical composition according to embodiment 69, wherein the sonosensitizer is indocyanine green.

What is claimed is:

1. A method of inducing cytokine secretion, the method comprising:
a. contacting mammalian antigen presenting cells (APCs) with a sonosensitizer and an immunomodulator; and
b. exposing the APCs of (a) to ultrasound radiation for a period of time sufficient to induce cytokine secretion by the APCs.

2. The method of claim 1, wherein the cytokine comprises one or both of IL-1β and TNF-α.

3. The method of claim 1, wherein the APCs comprise macrophages.

4. The method of claim 1, wherein the APCs are present in a mammalian subject.

5. The method of claim 4, wherein the mammalian subject has a tumor and contacting and exposing the APCs results in killing cells of the tumor.

6. A method of inducing secretion of IL-1β in a mammalian subject comprising
a. administering a sonosensitizer to the subject,
b. administering an immunomodulator to the subject, and
c. thereafter, exposing the subject to ultrasound radiation.

7. The method according to claim 1, wherein the immunomodulator comprises resiquimod (R848), PAPC, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, gardiquimod, 3M-052, SRL172, beta-glucan, Pam3Cys, Aquila's QS21 stimulon, vadimezan, AsA404 (DMXAA), a STING agonist, or a combination thereof, optionally wherein the immunomodulator comprises one or both of R848 and PAPC.

8. The method according to claim 7, wherein the immunomodulator is encapsulated in a liposome.

9. The method according to claim 7, wherein the immunomodulator is conjugated with a lipophilic moiety.

10. The method according to claim 9, wherein the lipophilic moiety is dioleoylphosphatidylethanolamine (DOPE) or cholesterol.

11. The method according to claim 1, wherein the sonosensitizer comprises a cyanine, porphyrin, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular and intermolecular charge-transfer dye or dye complex, tropone, tetrazine, bis(dithiolene) complexe, bis (benzene-dithiolate) complexe, iodoaniline dye, bis (S,O-dithiolene) complex, or a combination thereof, optionally, wherein the sonosensitizer comprises a cyanine.

12. The method according to claim 11, wherein the sonosensitizer is encapsulated in a liposome.

13. The method according to claim 11, wherein the sonosensitizer is conjugated with a lipophilic moiety.

14. The method according to claim 13, wherein the lipophilic moiety is dioleoylphosphatidylethanolamine (DOPE) or cholesterol.

15. A method of eliciting secretion of cytokines from immune cells in a mammalian subject comprising:
a. administering a sonosensitizer to the subject,
b. administering an immunomodulator to the subject,
c. thereafter, exposing the subject to ultrasound radiation.

16. The method according to claim 15, wherein the sonosensitizer comprises a cyanine, porphyrin, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine bis(dithiolene) complex, bis(benzene-dithiolate) complex, iodoaniline dye, and bis(S,O-dithiolene) complex, or a combination thereof.

17. The method according to claim 16, wherein the sonosensitizer is encapsulated in a liposome.

18. The method according to claim 16, wherein the sonosensitizer is conjugated with a lipophilic moiety.

19. The method according to claim 18, wherein the lipophilic moiety is DOPE or cholesterol.

20. The method according to claim 15, wherein the immunomodulator comprises resiquimod (R848), PAPC, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, gardiquimod, 3M-052, SRL172, beta-glucan, Pam3Cys, Aquila's QS21 stimulon, vadimezan, AsA404 (DMXAA), a STING agonist, or a combination thereof.

21. The method according to claim 20, wherein the immunomodulator is encapsulated in a liposome.

22. The method according to claim 20, wherein the immunomodulator is conjugated with a lipophilic moiety.

23. The method according to claim 22, wherein the lipophilic moiety is DOPE or cholesterol.

24. A method of promoting T cell activation in a mammalian subject comprising:
a. administering a sonosensitizer to the subject,
b. administering an immunomodulator to the subject, and
c. thereafter, exposing the subject to ultrasound radiation.

25. The method according to claim 24, wherein the sonosensitizer comprises a cyanine, porphyrin, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis(dithiolene) complex, bis(benzene-dithiolate) complex, iodoaniline dye, or bis(S,O-dithiolene) complex, or a combination thereof.

26. The method according to claim 25, wherein the sonosensitizer is encapsulated in a liposome.

27. The method according to claim 25, wherein the sonosensitizer is conjugated with a lipophilic moiety.

28. The method according to claim 27, wherein the lipophilic moiety is DOPE or cholesterol.

29. The method according to claim 24, wherein the immunomodulator comprises resiquimod (R848), PAPC, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, gardiquimod, 3M-052, SRL172, beta-glucan, Pam3Cys, Aquila's QS21 stimulon, vadimezan, AsA404 (DMXAA), a STING agonist, or a combination thereof.

30. The method according to claim 29, wherein the immunomodulator is encapsulated in a liposome.

31. The method according to claim 29, wherein the immunomodulator is conjugated with a lipophilic moiety.

32. The method according to claim 32, wherein the lipophilic moiety is DOPE or cholesterol.

33. A method of treating a tumor in a subject comprising:
a. administering a sonosensitizer to the subject,
b. administering an immunomodulator to the subject, and
c. thereafter, exposing the tumor to ultrasound radiation.

34. The method according to claim 33, wherein the sonosensitizer comprises cyanine, porphyrin, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis(dithiolene) complex, bis(benzene-dithiolate) complex, iodoaniline dye, and bis (S,O-dithiolene) complex, or a combination thereof.

35. The method according to claim 34, wherein the sonosensitizer is encapsulated in a liposome.

36. The method according to claim 34, wherein the sonosensitizer is conjugated with a lipophilic moiety.

37. The method according to claim 36, wherein the lipophilic moiety is DOPE or cholesterol.

38. The method according to claim 33, wherein the immunomodulator comprises resiquimod (R848), PAPC, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, gardiquimod, 3M-052, SRL172, beta-glucan, Pam3Cys, Aquila's QS21 stimulon, vadimezan, AsA404 (DMXAA), a STING agonist, or a combination thereof.

39. The method according to claim 38, wherein the immunomodulator is encapsulated in a liposome.

40. The method according to claim 38, wherein the immunomodulator is conjugated with a lipophilic moiety.

41. The method according to claim 40, wherein the lipophilic moiety is DOPE or cholesterol.

42. The method according to claim 1, wherein mammalian cells are human cells and the mammalian subject is a human.

43. A kit for inducing secretion of cytokines that promote T cell activation in mammals, the kit comprising:
a. a sonosensitizer comprising a cyanine, porphyrin, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular or intermolecular charge-transfer dye or dye complex, tropone, tetrazine, bis (dithiolene) complex, bis (benzene-dithiolate) complex, iodoaniline dye, bis (S, O-dithiolene) complex, or a combination thereof; and
b. an immunomodulator comprising resiquimod (R848), PAPC, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, gardiquimod, 3M-052, SRL172, beta-glucan, Pam3Cys, Aquila's QS21 stimulon, vadimezan, AsA404 (DMXAA), a STING agonist, or a combination thereof.

44. The kit according to claim 43, wherein either the sonosensitizer or the immunomodulator is encapsulated in a liposome.

45. The kit according to claim 43, wherein the sonosensitizer and the immune-modulator are both encapsulated in the same or in different liposomes.

46. The kit according to claim 43, wherein the sonosensitizer, the immunomodulator, or both are conjugated with one or more lipophilic moieties.

47. The kit according to claim 46, where the lipophilic moieties are selected from the group consisting of DOPE and cholesterol.

48. A pharmaceutical composition for parenteral administration to a subject comprising:
a. a sonosensitizer comprising a cyanine, porphyrin, merocyanine, phthalocyanine, naphthalocyanine, triphenylmethine, pyrilium dye, thiapyrilium dye, squarylium dye, croconium dye, azulenium dye, indoaniline, benzophenoxazinium dye, benzothiaphenothiazinium dye, anthraquinone, naphthoquinone, indathrene, phthaloylacridone, trisphenoquinone, azo dye, intramolecular and intermolecular charge-transfer dye or dye complex, tropone, tetrazine, bis (dithiolene) complexe, bis (benzene-dithiolate) complex, iodoaniline dye, bis (S,O-dithiolene) complex, or a combination thereof; and
b. an immunomodulator comprising resiquimod (R848), PAPC, CpG, polyIC, poly-ICLC, 1018 ISS, aluminum salts, Amplivax, AS15, BCG, CP-870, 893, CpG7909, CyaA, dSLIM, GM-CSF, IC30, IC31, ImuFact IMP321, IS Patch, ISS, ISCOMATRIX, Juv Immune, LipoVac, MF59, monophosphoryl lipid A (MPLA), Montanide IMS 1312, Montanide ISA 206, Montanide ISA 50V, Montanide ISA-51, OK-432, OM-174, OM-197-MP-EC, ONTAK, PepTel®, vector system, imiquimod, gardiquimod, 3M-052, SRL172, beta-glucan, Pam3Cys, Aquila's QS21 stimulon, vadimezan, AsA404 (DMXAA), a STING agonist, or a combination thereof; and
c. a pharmaceutically acceptable carrier.

49. The pharmaceutical composition according to claim 48, wherein the sonosensitizer or immunomodulator is encapsulated in a liposome.

50. The pharmaceutical composition according to claim 48, wherein the sonosensitizer and the immunomodulator are both encapsulated in the same or in different liposomes.

51. The pharmaceutical composition according to claim 48, wherein either the sonosensitizer or the immunomodulator or both are conjugated with one or more lipophilic moieties.

52. The pharmaceutical composition according to claim 51, where the lipophilic moieties are selected from DOPE or cholesterol.
